(19) United States
(12) Patent Application Publication
(10) Pub. No.: US 2009/0220564 A1
(43) Pub. Date: Sep. 3, 2009

(54) METHODS OF TREATING AND PREVENTING ACUTE MYOCARDIAL INFARCTION

(76) Inventors: William R. Baumbach, Hopewell, NJ (US); Hariharan Shankar, Highland Park, NJ (US); Oded Ben-Joseph, Sharon, MA (US)

Correspondence Address: PEPPER HAMILTON LLP, ONE MELLON CENTER, 50TH FLOOR, 500 GRANT STREET, PITTSBURGH, PA, 15219, US

(21) Appl. No.: 12/063,792

(22) PCT Filed: Aug. 18, 2006
(86) PCT No.: PCT/US06/32259

§ 371 Date: Aug. 28, 2008

Related U.S. Application Data
(60) Provisional application No. 60/710,009, filed on Aug. 19, 2005.

(51) Int. Cl. A61F 2/00; A61K 39/395

(52) U.S. Cl. 424/423; 424/136.1

(57) ABSTRACT

The present disclosure provides methods and compositions for treating coronary tissue damaged as a result of a cardiac disorder such as ischemia, acute myocardial infarction, vulnerable plaques, or reperfusion injury. Specifically, the cardiac disorder is treated using a multivalent molecule that is specific for a marker located, in or near the damaged coronary tissue and a marker located on a target cell (e.g., a stem cell). The multivalent molecule may be administered by intravenous injection, intra-arterial catheter, intramyocardial injection, or implantable device (e.g, stent).

### BACKGROUND OF THE INVENTION

This invention relates to methods and compositions for treating and preventing myocardial infarction through the use of multivalent molecules that assist in localizing therapeutic molecules and cells to the site of injury.

### BACKGROUND OF THE INVENTION

One of the greatest unmet clinical needs in interventional cardiology is amelioration or elimination of damage to heart tissue as a result of cardiac insult such as that caused by occluded coronary arteries—acute myocardial infarction (AMI). Not only does infarcted tissue decline due to lack of blood perfusion but, upon opening of the artery, the rapid reperfusion of the infarcted region also causes damage, typically in the form of cardiomyocytes undergoing apoptosis. In some instances, the long-term result of such damage is a permanent remodeling of ventricular tissue into a “scar” that is characterized by a thinning ventricular wall and expanded ventricular cavity. Often, the scar region does not contain sufficient cardiomyocytes and/or vascular cells, fibroblasts, or nerve cells to sustain the normal pattern of depolarization propagation and/or contraction for efficient pumping of blood. Reduced blood flow, associated with clinically measurable indications such as decreased contraction force, decreased ejection fraction, increased left ventricular (LV) volume, and increased LV wall stress, are the frequent sequela of AMI.

There are several clinical approaches to preventing AMI or reducing the damage caused by AMI, falling into three main categories: drug treatment; gene therapy; and cell therapy. Drug treatment includes delivery of heart medications such as beta blockers, nitrates, or calcium channel blockers; delivery of novel drugs intended to address specific modes of action, such as anti-apoptosis, modulation of differentiation, or cardio-protection; or delivery of potent peptide growth factors such as the pro-angiogenic VEGF or the anti-apoptotic IGF-I. Gene therapy includes delivery of DNA or viruses encoding genes, intended to induce angiogenesis or myogenesis, either systemically or locally into a coronary artery. Cell therapy includes surgically introducing cells directly into the heart muscle; injecting cells from inside the heart via catheter-mounted syringes using standard interventional techniques; introducing cells into a coronary artery, whence they would be carried by the blood downstream into the injured zone; or introducing cells intravenously. Other techniques include, for example, the enhancement of the healing properties of blood through super-oxygenization or other ex-vivo blood treatment.

Under the category of cell therapy, a variety of techniques have been used to select the specific cell type to introduce into a damaged region of the heart (Laflamme and Murray, Nature Biotech 23:845-856, 2005). One general approach consists of isolating autologous cells from tissues (e.g. skeletal muscle or bone marrow). These cells are then purified, altered, or expanded in such a way as to enhance the percentage of desired cell types prior to introduction into the AMI heart. This enhancement often is accomplished using conventional physical separation techniques such as density gradients; treatment with drugs or growth factors to promote or inhibit the expansion of specific cell types in culture; or using antibodies or other tethering molecule to physically identify and/or attract cells expressing specific surface markers. Another approach uses either autologous or non-autologous stem cells that have been isolated or purified from tissue or expanded and differentiated in cell culture prior to introduction.

Cell therapy approaches to AMI present many challenges. Technical difficulties include, for example, the inefficient injection of cells whereby only a few of the injected cells remain at the site of injection; the poor survival of transplanted cells; the expensive and time consuming processes for harvesting and ex vivo expansion of autologous cells; and the expensive clinical procedures required to introduce these cells into the heart of a patient. Other challenges to this approach include the choice of cell type or specific subtype that will be most beneficial when introduced into the damaged region of the heart.

There are several specific target regions that may be the subject of AMI therapies. The scar itself is considered to be less responsive to therapy because the endogenous tissue capable of functioning in coordination with the rest of the heart has been replaced by non-functional scar tissue cells and extracellular matrices. Tissue in areas surrounding the scar, called border regions, appears to be progressively more functional and thus more capable of incorporating new cells that could form or help to form functional tissue. Tissue that has undergone ischemia to some degree, but has not undergone apoptosis, necrosis, or scarring, is also a target for therapy insofar as treatment to slow or reverse the decline of this tissue via ischemia or reperfusion injury.

Many studies are underway or complete that address the treatment of AMI (Strauer et al, Circulation, 106:1913-1918, 2002; Assmus et al, Circulation, 106:3009-3017, 2002; Wollert et al, Lancet, 364:141-148, 2004; Perin et al, Circulation 110:11213-11218, 2004).

### SUMMARY OF THE INVENTION

The present in disclosure provides methods and compositions for treating cardiac disorders. In one aspect, the invention provides a method for treating a cardiac disorder in a patient by administering to the patient a molecule (e.g., a cardiovalent molecule) that is capable of binding (i) a coronary tissue marker and (ii) a target cell marker. Cardiac disorders amenable to treatment by this method include, for example, acute myocardial infarction, a chronic ischemic condition, reperfusion injury, chronic heart disease, vulnerable plaques, and cardiac fibrosis. Suitable molecules for use in this method include, for example, bispecific antibodies, bispecific F(ab′)₂ fragments, bispecific miniantibodies, diabodies, triabodies, and tetrabodies. Useful molecules include human IgG diabodies.

In useful embodiments, the molecule (e.g., cardiovalent molecule) is administered by intravenous injection, intra-arterial injection, intramyocardial injection, in the course of percutaneous transluminal coronary angioplasty, or using an implantable device (e.g., intra-arterial stent).

In another aspect, the invention provides a molecule (e.g., cardiovalent molecule) capable of binding (i) a coronary tissue marker and (ii) a target cell marker. Suitable molecules include a bispecific antibody, bispecific F(ab′)₂ fragment, bispecific miniantibody, diabody, triabody, and tetrabody. A particularly useful molecule is a human IgG.

In a related aspect, the invention provides a pharmaceutical composition containing the molecule described above (e.g., cardiovalent molecule) and a pharmaceutically acceptable carrier. In one embodiment, the pharmaceutically acceptable carrier is a microparticle. In another embodiment, the pharmaceutical composition is suitable for intravenous, intra-arterial, or intramyocardial injection.

In another related aspect, the invention provides an implantable device (e.g., a stent) containing the molecule described above (e.g., cardiovalent molecule). The molecule may be coated on the device or may be contained within a polymer coating on the device.

In one embodiment of any of the foregoing aspects, the molecule (e.g., cardiovalent molecule) is capable of binding coronary tissue including, for example, matrix metalloproteinase-1, matrix metalloproteinase-8, fibronectin, osteopontin, TLR4, IL1RL1-b, vascular cell adhesion molecule-1 (VCAM-1), Adam-like decysin-1 (ADAMDEC1), matrix metalloproteinase-9 (MMP-9), CD163, chemokine (C—X—C motif) ligand-2 (CXCL2), retinoic acid responder-1 (RARRES1), LpPLA2; colony-stimulating factor-2 receptor β (CSF2RB), cathepsin S, chemokine (C—C motif) ligand 18 (CCL18), spermidine/spermine N1 acetyltransferase, integrin β2 (ITGB2), cystatin B (CSTB), legumain; uncoupling protein 2 (UCP2), cathepsin B, RALGDS, F11 receptor (JAM-A), and ATPase plasma membrane 1 (ATP2B1).

In another embodiment of any of the foregoing aspects, the molecule (e.g., cardiovalent molecule) is capable of binding target cells including, for example, embryonic stem cells, umbilical cord blood stem cells, bone-marrow-derived stem cells, circulating stem cells, coronary stem cells, endothelial progenitor cells, and hormone/growth factor secreting cells.

In a related embodiment, the molecule (e.g., cardiovalent molecule) is capable of binding target cell markers including, for example, CD34, CD14, CD133, CXCR4, kinase insert domain receptor (KDR), Flk-1, VE-cadherin, c-Kit, and Sca-1.

In another embodiment of any of the foregoing aspects, the molecule (e.g., cardiovalent molecule) consists of a first V_{H} and V_{L} pair and a second V_{H} and V_{L} pair, wherein the first V_{H} and V_{L} pair has a different binding specificity compared to said second V_{H} and V_{L} pair. In useful embodiments, the molecule is derived from a hybridoma.

In another aspect, the invention provides a composition consisting of a target cell bound to a cardiovalent molecule. These compositions may also be used to treat cardiac disorders according to the principles of this disclosure.

By “cardiovalent molecule” is meant any multivalent molecule capable of binding at least (i) a coronary tissue marker and (ii) a target cell marker.

By “bispecific antibody” is meant a complete antibody molecule (usually IgG) in which the two antigen recognition regions (V_{L}-V_{H}) are different from one another. Bispecific antibody also refers to various engineered versions of IgG in which the antigen recognition regions are linked together through other means than by the traditional disulfide linkage, such as by chemical crosslinker, by peptide dimerization sequences, or as a single polypeptide chain. Bispecific antibody also includes structures that are smaller than full IgG, such as F(ab′)₂ and microantibodies (lacking all or part of the constant regions), yet containing two linked antigen recognition regions (Pluckthun and Pack, Immunotechnology, 3:83-105, 1997; Hudson, Curr Opin Immunol, 11:548-557).

By “diabody” is meant a bi-specific molecule derived from two antibody variable domains (V_{L}-V_{H}), usually in the form of single chain variable fragments (scFv), in which the antigen recognition regions of the variable domains are different from one another, and linked together either as a single polypeptide chain, by chemical crosslinkers, by virtue of the amino acid sequence within each individual scFv, or by any other suitable chemical or physical linkage such that the diabody is capable of binding to two independent antigens. Diabodies are described, generally, in U.S. Pat. No. 5,837,242.

By “triabody” is meant a multivalent molecule capable of binding two or three different antigens, in which three V_{L}-V_{H} regions, in the form of single chain variable fragments (scFv), are linked together as a single polypeptide chain, by chemical crosslinkers, by virtue of the amino acid sequence within each individual scFv, or by any other suitable chemical or physical linkage.

By “tetrabody is meant a multivalent molecule capable of binding two, three or four different antigens, in which four V_{L}-V_{H} regions, in the form of single chain variable fragments (scFv), are linked together as a single polypeptide chain, by chemical crosslinkers, by virtue of the amino acid sequence within each individual scFv, or by any other suitable chemical or physical linkage.

By “cardiac disorders” is meant any disease or disorder of the coronary tissue, particularly the cardiac muscle, associated with or caused by an ischemic condition, reduction in blood flow, physical trauma (e.g., associated with injury or a surgical procedure). Cardiac disorders include, but are not limited to, AMI, acute or chronic ischemic conditions, reperfusion injury, chronic heart disease (CHD), vulnerable plaques (VP), and cardiac fibrosis.

By “coronary tissue” is meant the cardiac muscle, consisting of fused cardiomyocytes, and ancillary cell types whose presence is critical, but are found in much smaller numbers. Examples of such cell types are vascular cells (including endothelial and smooth muscle cells), fibroblasts and other connective tissue cells, neurons and other types of nerve cells, and progenitor cells such as cardiac stem cells (Beltrami et al., Cell, 114:763-776, 2003). Coronary tissue also includes the extracellular matrix and associated molecules surrounding the foregoing cellular components.

By “coronary tissue marker” is meant any macromolecular marker (e.g., protein, lipid, and polysaccharide) that is expressed in coronary tissue following or in response to a cardiac disorder.

By “target cell” is meant any cell, including endogenous cells, autologous cells, heterologous cells, that provides therapeutic benefit for the treatment of a cardiac disorder. Target cells include, but are not limited to, embryonic stem cells, umbilical cord blood stem cells, circulating stem cells, bone marrow-derived stem cells, coronary stem cells, endothelial progenitor cells, and a hormone/growth factor secreting cells.

By “target cell maker” is meant any macromolecular marker (e.g., protein, lipid, and polysaccharide) that is expressed on the cell surface of a target cell and is useful to distinguish a target cell from at least some non-target cells.

By “percutaneous transluminal coronary angioplasty (“PTCA”) is meant a procedure for treating heart disease in which a catheter assembly having a balloon portion is introduced percutaneously into the cardiovascular system of a patient via the brachial or femoral artery or other available artery. The catheter assembly is advanced through the coronary vasculature until the balloon portion is positioned approximately across the occlusive lesion. Once in position, the balloon is inflated to a predetermined size to radially compress against the atherosclerotic plaque of the lesion to remodel the vessel. The balloon is then deflated to a smaller profile to allow the catheter to be withdrawn from the patient's vasculature.

By an “effective amount,” in reference to a therapeutic compound or composition, is meant an amount of a compound or composition, alone or in a combination according to the invention, required to affect a therapeutic response (i.e., treatment of cardiac disorders). The effective amount of active compound(s) used to practice the present invention for therapeutic treatment of cardiac disorders (e.g., AMI) varies depending upon the manner of administration, the age, body weight, and general health of the subject. Ultimately, the attending medical professional will decide the appropriate amount and dosage regimen. Such amount is referred to as an “effective amount.”

By “treating” is meant administering a pharmaceutical composition for the purpose of improving the condition of a patient by reducing, alleviating, or reversing at least one adverse effect or symptom.

### BRIEF DESCRIPTION OF THE DRAWINGS

FIG. 1 is a schematic diagram of common structures of bispecific antibodies and diabodies. The two different, specific antigen binding domains are shown in black and white, while a possible common light chain is shown in grey. FIG. 1A is a schematic diagram showing bispecific IgG, bispecific F(ab′)2, and diabody structures. FIG. 1B is a schematic diagram showing miniantibodies (lacking common chain regions), wherein the black or white lines denote linkages between variable light and variable heavy chains. The bispecific miniantibody contains two different subunits; whereas bispecific and bivalent miniantibody, containing two identical subunits.

FIG. 2 is a schematic diagram showing the various V_{H}-V_{L} combinations formed during the manufacture of a bi-specific (bivalent) molecule.

FIG. 3 is a schematic diagram showing components of a diabody-based system for the treatment of AMI. The bi-functional molecules are introduced by any convenient route (e.g., injection through a catheter into the artery feeding the infarcted zone, injected through a catheter directly into the cardiac wall in the infarcted zone, and/or eluted from a stent placed in the artery feeding the infarcted zone). Targets for site 1 of bi-functional molecule are contained within the infarcted zone and targets for site 2 of bi-functional molecule are found on circulating endogenous precursor cells or on cells within the heart.

### DETAILED DESCRIPTION OF THE INVENTION

The present invention provides methods and compositions for treating coronary tissue damaged as a result of a cardiac disorder such as ischemia, AMI, or reperfusion injury. Specifically, the cardiac disorder is treated using a multivalent molecule that is specific for a marker located in or near the damaged coronary tissue and a marker located on a target cell (e.g., a stem cell). The multivalent molecules may be administered by any appropriate method including, for example, via an implantable stent, via a drug-delivery PTCA balloon, as a locally injected bolus during PTCA, or directly into the cardiac wall in the infarcted region (i.e., intramyocardial injection). In one embodiment, the therapeutic strategy of this invention uses the multivalent molecule as a “bridging” molecule that binds at or near the damaged cardiac tissue and also binds a target cell that has therapeutic potential. Without being bound by any particular theory it is believed that the multivalent molecule causes the target cells to be retained in the region of cardiac damage for longer than the cells would otherwise remain and thus increases the likelihood, magnitude and duration of the therapeutic benefit conferred by the retained target cells. It is believed that target cells that are retained in the region of cardiac damage are therapeutically beneficial in one or more of the following ways: by differentiating into functional cardiac cell types such as cardiomyocytes or cardiovascular cells; by reducing the amount or degree of ischemic, inflammatory or reperfusion damage to the endogenous cardiac cells through secretion of beneficial hormones or growth factors; or by providing a paracrine benefit through secretion of hormones or growth factors that would in turn attract other beneficial cells, reduce the attraction for unwanted cell types, or limit the synthesis and accumulation of extracellular matrix, e.g. collagen, leading to cardiac fibrosis.

### Target Cells and Target Cell Markers

A variety of precursor cells that have the capacity to differentiate into coronary cells are believed to exist including, for example, bone marrow-derived cells, endogenous coronary stem cells, and endothelial progenitor cells (EPC). For circulating bone marrow-derived cells, known cell surface markers are expressed at various stages of differentiation into given cell types. For example, CD34+ cells are believed to differentiate into endothelial and myocardial lineages, and antibodies to CD34 are routinely used to isolate enriched populations of circulating bone marrow cells that are destined to enter these lineages (Verma et al, Circulation, 109:2058-2067, 2004; Szmitko et al, Circulation, 107:3093-4000, 2003). Thus, the binding domain of anti-CD34 antibodies is used as one functional moiety of a bi-functional molecule, to capture circulating cells with the capacity to repopulate damaged regions of the heart. It is believed that if the bi-functional molecule is anchored in the damaged region of the heart, it serves to isolate and restrain circulating precursor cells in the very region where they enhance the function of the damaged region. Additional cell surface markers, such as CD133 (Heiss et al, J Am Coll Cardiol, 45:1441-1448, 2005), CD14 (Romagnani et al, Circulation, 97:314-322, 2005; Leri and Kajstura, Circulation, 97 :299-301, 2005), CXCR4 (Ceradini et al, Nature Medicine, 10:858-864, 2004), kinase insert domain receptor (KDR) (Vasa et al, Circ Res, 89:e1-e7, 2001), Flk-1, VE-cadherin, (Martinez-Estrada et al, Cardiovasc Res, 65:328-333, 2005), c-Kit, or Sca-1 (Jackson et al, J Clin Invest, 107:1395-1402, 2001) could be used be used individually or in combination with CD34, with each other, or with other cell-surface markers to capture the most appropriate circulating or endogenous cells.

Cells that secrete hormones, growth factors, chemoattractants, or other ligands at the site of coronary disease or damage, such as AMI, would serve to enhance the function of endogenous coronary cells or attract coronary precursor or stem cells to the site of damage. For instance, the growth factors IGF-1 and HGF stimulate resident cardiac progenitor cells in canine infarcted hearts (Linke, Proc Natl Acad Sci USA, 102:8966-8971, 2005). Cells are identified that secrete such beneficial or protective factors, and specific surface markers are targeted by bi-functional molecules to tether such cells in the damaged region of the heart. Bi-functional molecules also serve to anchor autologous cells that have been engineered to secrete appropriate growth factors, in the precise region of coronary damage. In this case, CD34+ or other bone marrow derived cell type is transfected with human genes encoding growth factor(s) and associated control sequences to direct their secretion. Such cells, when introduced intravenously along with bi-functional molecules, are tethered in the region of coronary damage where they secrete appropriate factors that direct the expansion, growth, and/or differentiation of endogenous coronary cells.

### Cardiac Disorders and Markers

The matrix metalloproteinases (MMP)-1 and -8, fibronectin, and osteopontin (Kossmehl et al, J Mol Med, 2005) are cell surface markers that are expressed in damaged cardiac tissue, especially in response to ischemia and reperfusion. To anchor bi-functional molecules in the damaged region of a heart following ischemia and myocardial infarction, one functional moiety of such molecules would consist of specific binding sites that recognize, for example, MMP-1, MMP-8, fibronectin or osteopontin. Other markers that are selectively expressed on the surface of cells with the onset of AMI include, but are not limited to, IL1RL1-b (ST2 receptor) (Weinberg et al, Circulation, 106:2961-2966, 2002), and Toll-like receptors, e.g. TLR4 (Oyama et al, Circulation, 109:784-789, 2004).

Vulnerable plaque refers to a subgroup of high-risk coronary arterial plaques that result in acute coronary syndromes or sudden cardiac death, due to rupture followed by thrombosis and occlusion of the coronary artery. These plaques are not easily detected angiographically, and demonstrate the following characteristics: a thin, fibrous cap (<65 μm), a lipid-rich core, and increased macrophage activity. Awareness of vulnerable plaque has increased in recent years, with significant effort being devoted to development of a variety of detection methods such as intravascular ultrasound (IVUS), optical coherence tomography, intravascular magnetic resonance imaging, coronary spectroscopy, and intracoronary thermography (MacNeill et al, Arterioscler Thromb Vasc Biol, 23:1333-1342, 2003; El-Shafei and Kern, J Invas Card, 14:129-137, 2002). In addition, efforts are ongoing to establish the presence of soluble factors in blood, or serum markers, that would serve to identify the existence of VP which in turn predicts the onset of plaque rupture, which ultimately leads to the often fatal acute coronary syndrome (ACS). Several serum markers have been proposed to be used for this purpose: troponin-T, C-reactive protein, oxidized LDL, and soluble CD40 ligand, soluble ICAM-1, E-selectin, and soluble LOX-1. For purposes of treatment with bifunctional molecules, however, it is useful to target molecules that are exposed on the surface of the VP, either (a) on the surface of cells, such as smooth muscle cells and macrophages, that are found to be exposed to the serum or lumen in VP, or (b) as part of the extracellular matrix that forms the outer region of the fibrous cap of VP.

Examples of cell surface markers that serve this purpose are: CD40 ligand (Andre et al, Circulation, 106:896-899, 2002; Bavendiek et al, Arterioscler Thromb Vasc Biol, 25:1244-1249, 2005); lectin-like oxidized low-density lipoprotein receptor-1 (LOX-1) (Kataoka et al, Circulation, 99:3110-3117, 1999; Kataoka et al, Arterioscler Thromb Vasc Biol, 21:955-960, 2001; Kume and Kita, Circ Res, 94:269-270, 2004); pregnancy-associated plasma protein A (PAPP-A) (Cosin-Sales et al, Circulation, 109:1724-1728, 2004; Heeschen et al, J Am Coll Cardiol, 45:229-237, 2005; Fichtlscherer et al, Curr Opin Pharmacol, 4:124-131, 2004); and monomeric (or modified) C-reactive protein (mCRP) (Schwedler et al, Circulation, 112:1016-1023, 2005).

Examples of extracellular matrix markers that are believed to allow targeting of the fibrous cap of VP are fibrillar collagen types I and III, specifically cleaved interstitial collagen (Horton et al, Ann NY Acad Sci, 947:329-336, 2001); metalloproteinases that specifically target collagen, thus rendering the fibrous cap to be thinner and more prone to rupture, such as MMP-1, MMP-8 and MMP-13 (Horton et al, Ann NY Acad Sci, 947:329-336, 2001)), and vascular cell adhesion molecule-1 (VCAM-1) (Vasan, Circulation, 113:2335-2362, 2006). In addition, differential expression analysis has identified molecules with higher mRNA levels, higher protein levels, or both, in unstable plaque compared to stable plaque specimens (Papaspyridonos et al, Arterioscler Thromb Vasc Biol, 26:1837-1844, 2006). These molecules are useful target cell markers to which the therapeutic cardiovalent molecules of this invention may be directed. These target cell include Adam-like decysin-1 (ADAMDEC1); MMP-9; CD163; chemkine (C—X—C motif) ligand-2 (CXCL2); retinoic acid responder-1 (RARRES1); LpPLA2; colony-stimulating factor 2 receptor-β (CSF2RB); cathepsin S; chemokine (C—C motif) ligand 18 (CCL18); spermidine/spermine N1 acetyltransferase (SAT); integrin β2 (ITGB2); cystatin B (CSTB); legumain; uncoupling protein 2 (UCP2); CYP1B1; cathepsin B; RALGDS; F11 receptor (JAM-A); and ATPase plasma membrane 1 (ATP2B1).

Attracting cells with the capacity to create or enhance endothelial coverage of such regions of VP are believed to determine the second binding site for the bifunctional molecule. Thus, the commonly used marker for endothelial progenitor cells (EPC), CD34, is a useful target marker (Verma et al, Circulation, 109:2058-2067, 2004). Additional cell surface markers, such as CD133 (Heiss et al, J Am Coll Cardiol, 45:1441-1448, 2005), CD14 (Romagnani et al, Circulation, 97:314-322, 2005; Leri and Kajstura, Circulation, 97 :299-301, 2005), CXCR4 (Ceradini et al, Nature Medicine, 10:858-864, 2004), kinase insert domain receptor (KDR) (Vasa et al, Circ Res, 89:e1-e7, 2001), Flk-1, VE-cadherin, (Martinez-Estrada et al, Cardiovasc Res, 65:328-333, 2005), c-Kit, or Sca-1 (Jackson et al, J Clin Invest, 107:1395-1402, 2001) is used individually or in combination with CD34 to attract a population of circulating cells that effectively provides a layer of EPC, endothelial cells (EC), or similar cell type capable of promoting the healing and stabilization of vulnerable plaque.

Thus, without being bound by any theory, it is believed that the two functional moieties of the bi-functional molecule together recognize and hold appropriate target cells in the damaged region of the heart by (a) recognizing a marker in the damaged region of the heart and (b) recognizing a marker on target cells that either differentiate into functioning cardiac cells and/or otherwise improve the function of existing cardiac cells To accomplish this efficiently, such bi-functional molecules are delivered directly to the site of cardiac damage (e.g., from a drug-eluting stent or injection during PTCA) or systemically (e.g., by intravenous or intra-arterial injection).

A variety or laboratory tests are available to identify other markers of cardiac disorders. For instance, differential cDNA analysis identify proteins whose expression is upregulated upon injury or damage; among these, known or suspected cell-surface proteins to which antibodies are useful as targets for the purpose of this invention. Extracellular matrix (ECM) proteins are often uniquely found in particular tissues, or in tissues under particular circumstances, e.g. stress or damage; such unique proteins will also serve as targets for the purpose of tethering desired precursor cells to damaged heart tissue. Similarly, cell surface markers that are found on known or suspected precursors of functional cardiomyocytes (or other beneficial coronary cells) serve as targets for antibodies or other molecules. Thus, one identified binding site is usefully found on or near cells in the damaged heart, and another identified binding site is usefully found on precursor cells capable of differentiating into healthy, functioning coronary cells. The structural moieties from antibodies that bind to these targets are the raw material used to create bifunctional molecules, e.g. diabodies, whose purpose is to bring the two cell types into proximity, and to ultimately repopulate the damaged area of the heart with functioning cells.

### Therapeutic Multivalent Molecules

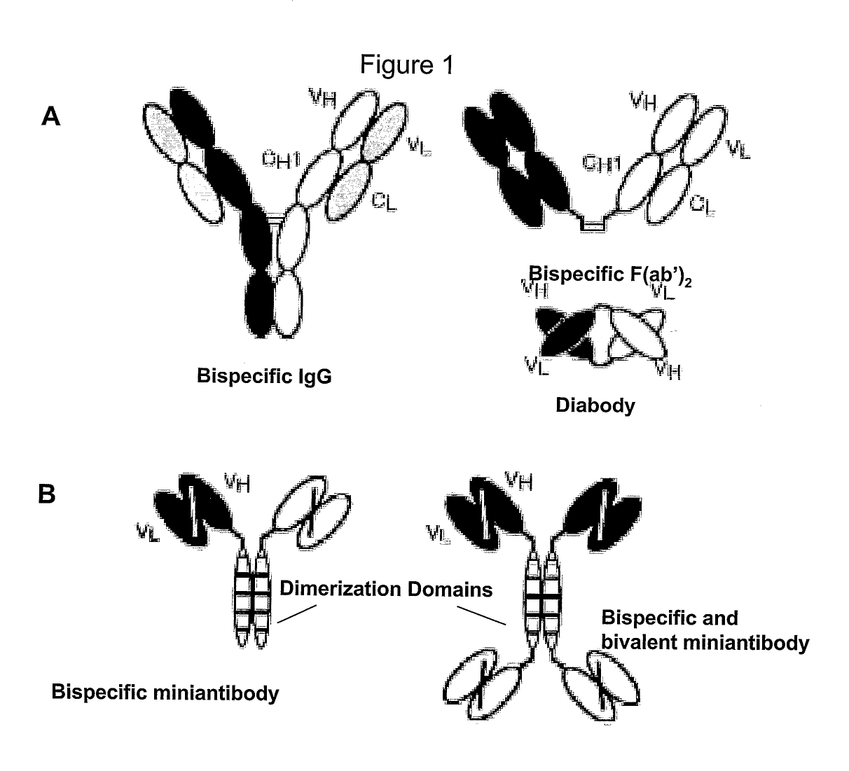

Any suitable multivalent molecule that is at least bi-specific for a coronary tissue marker and a target cell marker is contemplated. Examples of such multivalent molecules include chimeric antibodies, bispecific antibodies, bispecific F(ab′)₂ fragments, bispecific miniantibodies, diabodies, triabodies, tetrabodies, or other bi-functional or multi-functional organic molecules. FIG. 1 is a schematic diagram showing several types of bivalent molecules that are useful in accordance with the principles of this disclosure. Other types of multivalent molecules useful in the present invention are described more fully in U.S. Pat. No. 5,837,242.

In one embodiment, the bifunctional molecule is a diabody that attaches to regions of the heart downstream from an injured coronary artery receiving PTCA, and also attaches to surface markers on cells in the circulation or interstitial spaces capable of either repopulating the heart with functional myocardial or other beneficial cells, or otherwise enhancing heart function.

Diabodies, or bifunctional antibodies, are generally considered to be engineered versions of an IgG molecule, whereby each functional half consists of a heavy and light chain whose origin is a monoclonal antibody, either directly from a mouse hybridoma or a humanized version in which the non-antigen binding portions have been replaced with the corresponding regions from human genes. The two different functional halves of the diabody are either joined together in vivo in an antibody producing mammalian cell, or are joined in vitro from individually produced single chains made in a bacterial system such as E. coli.

Bifunctional molecules are produced that contain two independent protein-binding fragments combined into a single polypeptide chain. For example, a single-chain antibody variable region can be genetically combined with another protein such as staphylococcal protein A such that both protein binding functionalities are expressed as a single polypeptide chain in E. coli (Tai et al., Biochemistry, 29:8024-8030, 1990).

Bifunctional molecules, as well as higher order multi-valent molecules such as triabodies and tetrabodies, are engineered from the basic building blocks of antibodies. The single chain variable fragment (scFv) is the smallest polypeptide that contains the complete antigen binding region of an antibody, and are produced in a bacterial system such as E. coli (Huston et al., Methods Enzymol., 203:46-88, 1991). This methodology is useful for bivalent molecules, since the molecular weight (approximately 60 kDa) of two scFv is substantially less than that of a diabody resembling an intact IgG molecule (containing both heavy and light chains). In some embodiments, the reduced molecular weight provides an advantage in formulation as a device coating because thin coatings are typically more biocompatible than thick coatings, and generally less material is required to achieve a similar molar concentration as IgG-like diabodies. Two, three or four scFv may be combined into single molecules through the use of peptide linkers, whereby the length and structure of the linkers can determine the valence of the final multi-functional molecule (Kortt et al., Biomol. Engineering, 18:95-108, 2001). One embodiment is a bifunctional molecule. It is also contemplated that higher order valences offer enhanced binding properties such as binding avidity or cell type specificity, depending on the specificity and orientation of the binding functionalities.

Non-antibody therapeutics such as multi-functional organic molecules are contemplated. These molecules consist of peptides on the order of 12-20 amino acids, each of which is selected for high affinity binding to target sequences, using a methodology such as phage display, and subsequently combined using peptide linkers (Rajagopal et al., Bioorg. Med. Chem. Lett., 14:1389-1393, 2004).

Formulation of Multivalent Molecules

The multivalent molecules used for treating a cardiac disorder are formulated in a variety of ways depending on the route of administration. For direct injection intravenously, a saline solution of the therapeutic molecule are useful. Alternatively, if the molecule is insoluble in saline, formulation in other non-toxic physiologically acceptable carriers, adjuvants, or vehicles for parenteral injection are useful. Pharmaceutical compositions according to the present invention also comprise binding agents, filling agents, lubricating agents, disintegrating agents, suspending agents, preservatives, buffers, wetting agents, and other excipients. Examples of filling agents are lactose monohydrate, lactose hydrous, and various starches; examples of binding agents are various celluloses, including low-substituted hydroxylpropyl cellulose, and cross-linked polyvinylpyrrolidone; an example of a disintegrating agent is croscarmellose sodium; and examples of lubricating agents are talc, magnesium stearate, stearic acid, and silica gel. Examples of suspending agents are hydroxypropyl cellulose, methyl cellulose, hydroxyethyl cellulose, carboxymethyl cellulose sodium, hydroxypropyl methylcellulose, acacia, alginic acid, carrageenin, and other hydrocolloides. Examples of preservatives, which control microbial contamination, are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

For local injection via catheter at the site of PTCA, into the coronary artery lumen, similar formulations as above are useful. In addition, therapeutic molecules may be encapsulated in microparticles or nanoparticles, typically between 100 nm and 1000 nm in diameter, that are believed to serve to delay release of the molecules into the surrounding serum or tissue. In addition, microparticles or nanoparticles containing bifunctional molecules are taken up by cells in the region of the target tissue, thus improving the efficiency of delivery. Examples of materials used to form microparticles or nanoparticles are poly lactic acid, poly(D,L-lactide-co-glycolide) (PLGA), liposomes, and dextran (Jiang et al, Adv Drug Deliv Rev, 57:391-410, 2005; Bala et al, Crit Rev Ther Drug Carrier Syst, 21:387-422; Kayser et al, Curr Pharm Biotechnol, 6:3-5, 2005; U.S. Pat. No. 6,805,879; U.S. Patent Application 20040191325).

For local delivery from stents or other implanted devices from which the flow of blood carry eluted substances downstream to the target tissue, bifunctional molecules are mixed with polymers (co-dissolved or emulsified) and coated on such devices by spraying or dipping. The polymer is typically either bioabsorbable or biostable. A bioabsorbable polymer breaks down in the body and is not present sufficiently long after implantation to cause an adverse local response. In some embodiments, bioabsorbable polymers are gradually absorbed or eliminated by the body by hydrolysis, metabolic process, bulk erosion, or surface erosion. Examples of bioabsorbable materials include but are not limited to polycaprolactone (PCL), poly-D, L-lactic acid (DL-PLA), poly-L-lactic acid (L-PLA), poly(lactide-co-glycolide), poly(hydroxybutyrate), poly(hydroxybutyrate-covalerate), polydioxanone, polyorthoester, polyanhydride, poly(glycolic acid), poly(glycolic acid-cotrimethylene carbonate), polyphosphoester, polyphosphoester urethane, poly(amino acids), cyanoacrylates, poly(trimethylene carbonate), poly(iminocarbonate), copoly(etheresters), polyalkylene oxalates, polyphosphazenes, polyiminocarbonates, and aliphatic polycarbonates. Biomolecules such as heparin, fibrin, fibrinogen, cellulose, starch, and collagen are typically also suitable. Examples of biostable polymers include parylene, polyurethane, polyethylene, polyethlyene teraphthalate, ethylene vinyl acetate, silicone and polyethylene oxide (PEO). Bifunctional molecules may also be coated on stents or other devices on which grooves, holes, a micropores or nanopores have been engineered into the surface, such that the formulation to be delivered is sequestered within the pores and released slowly into the lumen of the coronary artery after the device has been implanted.

### Administration of Multivalent Molecules

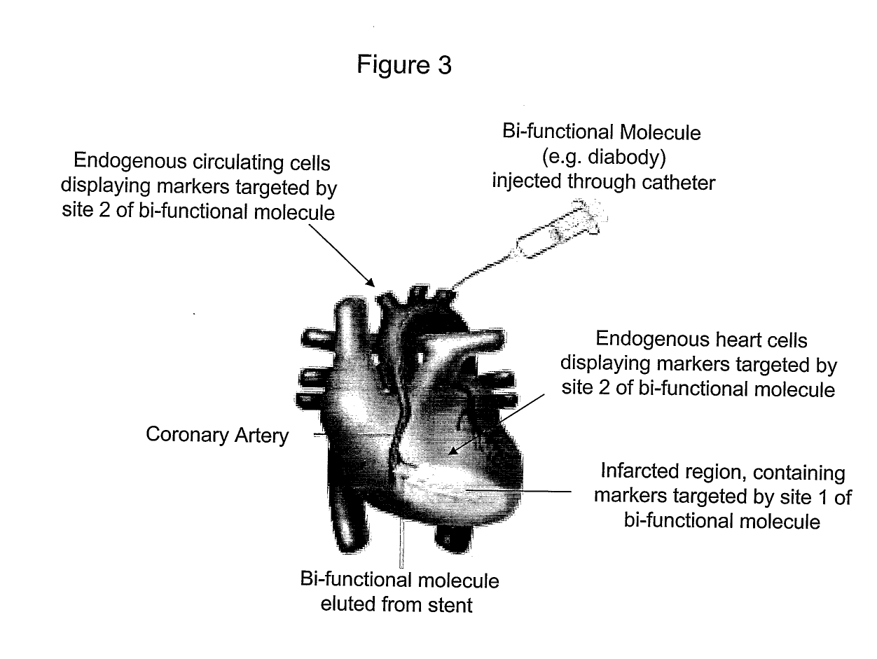

The multivalent molecules used for treating a cardiac disorder are administered by any appropriate method. Methods for administration include, for example, intravenous injection, a bolus injection via a catheter during PCTA, a coated or impregnated implantable device such as a stent, and injection directly into the target tissue, i.e. in and around the infarcted region of the heart (see, for example, FIG. 3). Examples of implantable devices and stents of the present invention include, but are not limited to, those described in U.S. Pat. Nos. 6,709,379, 6,273,913, 5,843,172, 4,355,426, 4,101,984, 3,855,638, 5,571,187, 5,163,958 and 5,370,684; U.S. Patent Publications US2002/0098278 and US2004/0073284; PCT International Published Patent Application No. WO 2004/043292; and European Published Patent Application No. EP 0875218.

Delivery of bifunctional molecules upstream of the site of AMI, from which point they are carried by the flow of blood to the site of AMI, is accomplished through standard interventional devices including, but not limited to, drug-eluting stents, drug-delivery balloons, or PTCA catheters. In each case, the molecules are ultimately delivered into the lumen of the coronary artery whose occlusion resulted in hypoxia and infarction. Therapeutic molecules are also formulated in a variety of conventional pharmaceutical carriers including saline, emulsifiers, or microparticles. Additionally, the molecules may be coated on a coronary stent either alone or embedded in polymers or other natural or synthetic carriers, for controlled elution into the lumen of the coronary artery. In every case, the therapeutic molecules will be taken via the coronary arterial system into the region of the heart that was subject to damage by ischemia. Typically, the same coronary artery lesion that is undergoing treatment by PTCA and/or stenting is the cause of the arterial blockage that would lead to AMI due to ischemic and/or reperfusion injury. Delivery of therapeutic molecules into the arterial lumen at the site of intervention will thus effectively provide treatment to the precise site of coronary damage.

In one embodiment, bifunctional molecules are delivered via one or both of two methods during the same interventional procedure, typically but not limited to PTCA. The first is via catheter-based injection of therapeutic molecules (formulated in one or more ways, such as in saline, in a pharmaceutical excipient, or encapsulated in microparticles or nanoparticles) directly into the coronary arterial lumen, in the region of PTCA and stent placement, using the balloon catheter itself or a separate catheter (Guzman et al, Circulation, 94:1441-1448, 1996). The second delivery method is via a coating on the coronary stent. Here, at least one therapeutic compound is embedded in a degradable or non-degradable polymer or natural coating, or alternatively formulated as microparticles which are themselves embedded in a polymeric or other type of coating, and subsequently these coatings are applied to the surface of the coronary stent. Bifunctional molecules elute from the coating into the lumen of the coronary artery either by diffusion from the polymeric coating or by degradation of the coating, thus releasing the therapeutic molecules. For local delivery by drug delivery balloons, the bifunctional molecules in a liquid formulation can be loaded into devices such as a triple-lumen balloon catheter (Infiltrator, manufactured by InterVentional Technology; Kaul et al, Circulation, 107:2551-2554, 2003), or a channel balloon catheter (Boston Scientific) from which the drug is injected into the arterial wall, and thence released into the arterial lumen, from where it would flow into the coronary infarcted zone.

For local delivery directly into the target tissue, i.e. into the coronary ventricular wall in and around the infarcted zone, bifunctional molecules in a liquid formulation are injected from an intramyocardial injection catheter (Boston Scientific Corporation Stiletto™ endocardial direct injection catheter system; Marshall et al, Molecular Therapy, 1:423-429, 2000; Karmarkar et al, Magnetic Resonance in Medicine, 51:1163-1172, 2004).

Bifunctional molecules are usefully presented to the target region at the most optimal time, i.e. upon reperfusion after balloon angioplasty. Many, if not all, situations in which a blocked artery is re-opened via PTCA result in some level of reperfusion injury. Introduction of bi-functional molecules enhances the repopulation of such injured regions with beneficial cells, thus reducing the extent or duration of injury.

### Example 1

### Differential cDNA Analysis

To target multivalent molecules to regions of ischemic or reperfusion injury in the heart, specific cell surface or extracellular markers are identified through the analysis of diseased human tissue in comparison with healthy tissue. Many conventional methods have been developed to detect differential expression of genes in the cardiovascular system, leading to identification of specific proteins of interest (reviewed in Napoli et al, Heart 89:597-604, 2003). These are typically based on comparisons of messenger RNA levels (sometimes in the form of cDNA) in healthy versus diseased tissue samples. Commercial databases are also available that provide such information (see U.S. Patent Application 2003/0129624).

Briefly, small samples of tissue are dissected from the hearts of human cadavers. One set of samples is taken from the left ventricular wall of a healthy heart. Other sets of samples are taken from the left ventricular wall of hearts that have undergone ischemia and/or AMI. Samples are characterized by length of time post ischemia (if known), region of sample with respect to infarct zone, severity of infarct, age of donor, gender of donor, and other disease states of donor. Samples are flash frozen in liquid nitrogen upon dissection. The frozen tissue is crushed, homogenized, and Poly-A+ RNA is extracted from the tissue and purified according to standard procedures (Chirgwin et al, Biochemistry, 18:5294, 1979) and exemplified in e.g. GeneChip® Expression Analysis Technical Manual, Affymetrix, 2004. Poly A+ RNA is converted enzymatically to cDNA or cRNA using standard procedures and may be labeled with biotin prior to hybridizing to a microarray consisting of synthetic single stranded DNA fragments derived from the sequences of expressed human genes, e.g. the Affymetrix Human Genome U133 GeneChip® array. Microarrays are hybridized with labeled cDNA or cRNA for e.g. 24 hours at 45° C., and stained with e.g. Streptavidin phycoerythrin (Molecular Probes) in an automated fluidic station or similar device, then scanned for fluorescent signals by e.g. the Hewlett-Packard GeneArray™ scanner. Data from such analyses is integrated, stored, and analyzed using software such as the Affymetrix Microarray Suite (v. 5.0). In such a way, genes that are differentially expressed in healthy heart tissue vs. ischemic or infarcted heart tissue are identified. Proteins that are expressed from these genes are candidates for use as specific markers of AMI or other heart conditions to be targeted by bifunctional molecules, i.e. proteins made by diseased tissue and not by healthy tissue would be preferred target candidates.

Alternatively, commercial products such as the ASCENTA® system (GeneLogic, Gaithersburg, Md.) provide expression data from healthy and diseased tissues that have been pre-screened and analyzed. Thus, the search for differentially expressed genes or confirmation of expression data for prospective gene candidates may be greatly simplified. In addition, proprietary databases or proprietary annotation of public databases may afford the ability to identify the likely cellular compartment in which gene products of interest may be found. For the purpose of enhancing the retention of target cells in diseased areas of the heart, it is likely that proteins, or portions thereof, that are exposed on the cell surface or in an extracellular compartment would be most useful because they would be readily accessible to binding by multivalent molecules.

Once genes of interest have been identified that encode suitable cognate proteins, such proteins must be obtained in sufficient quantities to enable the production of monoclonal antibodies, or to use for screening one of many possible antibody or antibody-like libraries. In many cases, this has been previously accomplished so that suitable protein and/or antibodies are already commercially available, e.g. anti-CD34+ antibodies (Szmitko et al, Circulation, 107:3093-3100, 2003). If the proteins in question are not readily available, a variety of methods may be used to produce them. Using standard cloning techniques, the appropriate gene transcripts can be cloned into commercial vectors for expression and production of the proteins in mammalian, avian, insect, prokaryotic, or cell-free systems. Many contract research organizations, e.g. Promega, Madison, Wis., specialize in such activities.

### Example 2

### Multivalent Antibody Production

Multivalent antibodies are one type of multivalent molecule that is used in accordance with the principles of this disclosure. A bispecific antibody (diabody) displays high affinity binding and specificity for two different biological markers. The components for diabodies may be produced in a similar way as traditional monoclonal antibodies, e.g. in hybridomas (tissue culture cells that have been induced to produce a single antibody in vitro) or in engineered bacteria, and the components recombined in vitro to produce chimeric molecules Alternatively, a single hybridoma producing two different monoclonal antibodies are engineered whereby a fraction of the resulting antibodies are diabodies. For human clinical use, human or humanized antibodies are required to avoid the development of a human anti-mouse antibody response. Thus, human or humanized antibodies (Vaughan et al, Nat. Biotechnol. 16:535-539, 1998) are first be identified that recognize the antigens of choice, e.g. cell-surface protein markers displayed on two different cell types. This has been achieved by: creating chimeric mouse antibodies with substantial portions derived from human genes; isolating human antibodies using phage display technology; or producing hybridomas from transgenic mice expressing a substantial component of the human Ig gene locus.

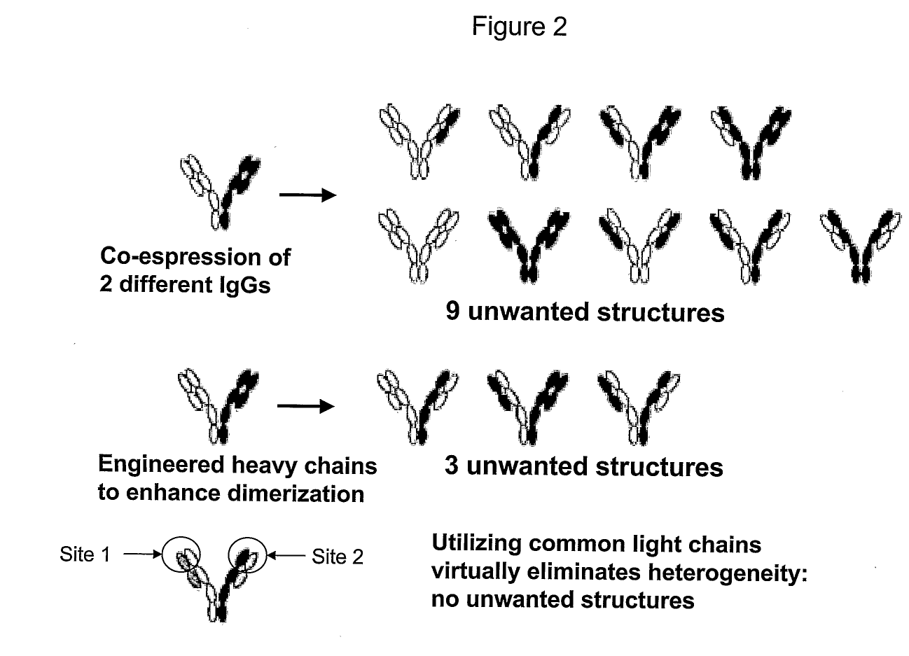

To produce sufficient quantities of a diabody such that pre-clinical and clinical trials can be performed, highly efficient production and purification methods is employed. The coexpression of two different IgG is inefficient due to the unwanted pairings of component heavy and light chains that are either made in the same cell, or combined in vitro from material produced in different cells. One approach (reviewed in Carter, J Immunol Methods, 248:7-15, 2001) entails a series of bioengineering steps that allows the components of each IgG unit to be secreted from E. coli and combined in vitro in such a way that a single preferred structure is produced in a relatively high proportion. The essential approach of this method is illustrated in FIG. 2. Based on the structure of two independent antibodies, each antibody consisting of two light and two heavy chain domains, ten (10) different heterodimeric structures would be possible, thus complicating the isolation of the desired structure (see FIG. 2). To enhance the formation of heterodimers between heavy chains over that of homodimers, thus reducing the number of possible combinations to four (4), portions of the heavy chain gene are engineered to enhance the interaction between different heavy chains while reducing the interaction between identical heavy chains (Merchant et al, Nat. Biotechnol. 16:677-681, 1998). Finally, the mispairing of antibody light chains is circumvented through the use of a single antibody light chain, isolated so as to not interfere with the specificity of heavy chain recognition of its cognate antigen (Vaughn et al, Nat. Biotechnol. 14:309-314, 1996), thus enabling the production of a single preferred combination of components.

Other methods for producing diabodies have been described in the technical and patent publications including, for example, Suresh et al, Methods Enzymol. 121:210-228, 1986, and U.S. Pat. Nos. 5,837,242, 5,807,706, and 5,648,237.

### Example 3

### Treatment of AMI in a Porcine Model

Female or castrated male juvenile hybrid farm swine, 10-16 weeks old and weighing 35+/−10 kg, are utilized. Fasting is conducted prior to induction of anesthesia for device deployment, sample collection for serum chemistry and necropsy. Food, but not water, is withheld the morning of the procedure. To prevent or reduce the occurrence of thrombotic events, anti-platelet pharmacological therapy consisting of clopidogrel (75 mg per os [PO]) and acetylsalicylic acid (ASA; 325 mg, PO) is administered daily, with the exception of the implantation day, beginning at least 3 days prior to the scheduled procedure date.

Animals are tranquilized using intramuscular ketamine, azaperone or acepromazine and atropine. Anesthesia induction is achieved with propofol injected intravenously [IV] through a catheter in a peripheral ear vein. Upon induction of light anesthesia, the subject animal is intubated and supported with mechanical ventilation. Isoflurane (1 to 5.0% to effect by inhalation) in oxygen is administered to maintain a surgical plane of anesthesia. Prophylactic antibiotic duplocillin LA® 0.05 ml/kg is given intramuscular [IM]. Intravenous fluid therapy is initiated and maintained throughout the procedure with saline (1 ml/kg/hour). The rate may be increased to replace blood loss or low systemic blood pressure.

The animal is placed in dorsal recumbency, and hair removed from access areas. Animals are kept warm throughout the preparation and the procedure. Limb-leads are placed, and electrocardiography established. The access site is prepared with chlorexidine, 70% isopropyl alcohol and proviodine, and the area is appropriately draped to maintain a sterile field. After animal preparation, the femoral artery is accessed using a percutaneous approach. Alternatively, an incision is made in the inguinal region to expose the femoral artery. An infiltration of bupivacain 0.5% (5 ml IM) on the femoral access site is performed to achieve local anesthesia and manage pain after surgery. A 7F or 8F-introducer arterial sheath is introduced and advanced into the artery. The sheath is connected to a pressure transducer for monitoring arterial pressure. An initial bolus of heparin (400 U/kg IV) is given and ACT performed approximately 5 minutes later. If ACT is under 300 seconds, an additional 100 to 400 U/kg of heparin is given. ACT is tested approximately every 20 minutes.

Under fluoroscopic guidance, a 7F guide catheter is inserted through the sheath and advanced to the appropriate location. After placement of the guide catheter, angiographic images of the coronary vessels are obtained with contrast media to identify the proper location for the deployment site. Quantitative angiography will be performed after injection of nitroglycerin 500 μg intracoronary [IC] to determine the appropriate vessel size for implantation and/or occlusion.

After visualization of the coronary artery anatomy, a segment of artery ranging from 2.6 mm to 3.5 mm mid-segment diameter is chosen, and a 0.014″ guidewire is inserted into the chosen artery. QCA is performed to accurately document the reference diameter for balloon angioplasty and/or stent placement.

Each stent delivery system or balloon catheter is prepared by applying vacuum to the balloon port; contrast/flush solution (50:50) is then introduced by releasing the vacuum. The appropriately sized balloon is introduced into the appropriate artery by advancing the balloon catheter through the guide catheter and over the guidewire to the deployment site. The balloon is then inflated at a steady rate to a pressure sufficient to target a balloon:artery ratio of 1.1:1 (acceptable range of 1.05-1.15:1) and held for 60 minutes. A contrast injection is performed during full inflation to demonstrate occlusion with the balloon. During occlusion, monitoring is performed for heart functions, and other monitoring parameters include isoflurane level, SaO₂, pulse rate, blood pressure, temperature, O₂ flow, and tidal volume. After the occlusion period, vacuum is applied to the inflation device in order to deflate the balloon. Complete balloon deflation will be verified with fluoroscopy.

Immediately upon reperfusion, the test treatment is initiated. In one embodiment, a coated stent containing a bi-functional molecule is placed (target balloon:artery of 1.15:1) in the same artery and region as the occlusion balloon. In a second embodiment, the bi-functional molecule is administered through the balloon catheter (or other injection catheter) into the region of the balloon occlusion. In a third embodiment, a drug delivery balloon is utilized to introduce the bi-functional molecule into the region of the balloon occlusion. In a fourth embodiment, the bi-functional molecule is administered via intramyocardial injection into the infarcted region of the heart. A useful embodiment would involve a bolus injection via catheter while simultaneously providing the same, similar, or complementary bi-functional molecule in a stent coating, which would elute over a period of hours, days, or weeks.

Following completion of angiography, all catheters and sheaths are removed. If percutaneous access is achieved, pressure is applied to the access site until hemostasis is obtained. If a cutdown is performed, the femoral artery is ligated. The incision is closed in layers with appropriate suture materials.

The animals are placed in a pen and monitored during recovery from anesthesia for four to five hours following the procedure. Medical treatment including analgesia is given as needed. Animals in apparent severe pain or distress, as determined by clinical observation and consultation with the facility veterinarian is euthanized. ASA (325 mg/day PO) and clopidogrel (75 mg/day PO) are administered for the duration of the study. Morbidity/mortality checks and clinical observations are performed twice daily.

At the designated endpoint, typically 6 weeks post procedure, the animals are analyzed by MRI for LV function. Animals are then euthanized, the heart is excised, and the atria and great vessels are trimmed away. Next, the RV free wall is trimmed away from the LV (with septum intact). The LV is blotted dry, weighed and indexed by body weight (in kg). The LV is sectioned transversely into five equal segments from apex to base, immersed in 10% buffered formalin, dehydrated at room temperature through ethanol series, and embedded in paraffin.

Serial 5-μm sections are prepared using a standard microtome. Sections are mounted and stained with hematoxylin and eosin (or trichrome) for determination of infarct size. Quantitative histological analyses are performed, and infarct size is determined (Pfeffer et al, Circulation, 81:1161-1172, 1990). Infarct length is measured along the endo- and epicardial surfaces from each of the five LV segments (three sections per segment). Total LV circumference is measured along the endo- and epicardial surfaces from each of the five LV segments (three sections per segment). Infarct size is determined as percentage of total LV circumference. The ratio of scar length to body weight is calculated to exclude the potential influence of differences in body weight on infarct size.

Remodeling parameters. The maximum longitudinal dimension is measured before left ventricular sectioning. The LV is sectioned transversely into five equal segments from apex to base, and the maximum short-axis dimension after sectioning is measured. Outlines of the section rings and infarct scars are made on plastic overlays. “Thinning” ratio (ratio of average thickness of infarcted wall to average thickness of the normal wall) is measured by computerized planimetry. The maximum depth of infarct scar bulge in millimeters is measured on the contoured sections as an index of regional dilation. Bulging normalized to body weight is calculated. Ventricular volumes are computed from the short-axis areas and the long-axis length by the modified Simpson's rule, as used for echocardiographic studies during remodeling (Jugdutt et al, Circulation, 89:2297-2307, 1994).

The remaining sections are stained with Sirius red F3BA (0.1% solution in saturated aqueous picric acid) to discriminate between cardiomyocytes and collagen matrix (Wollert et al, Circulation, 95:1910-1917, 1997). Volume collagen fraction is calculated as the sum of all connective tissue areas divided by the total area of the image.

### Example 4

### Delivery of Bi-Functional Molecules from Microparticles and/or Coronary Stents

### Therapeutic Dosage

Therapeutically effective amounts of a bi-functional molecule are administered via intra-arterial injection, intravenous injection, intramyocardial injection, PTCA catheter, PTCA balloon, or indwelling device such as a stent. It is contemplated that bi-functional molecules is administered simultaneously by one or more of these methods, e.g. in a coating on a coronary stent and additionally injected through a PTCA catheter. Thus, the dosage is delivered both immediately by injection and over a prolonged period by elution from a stent. Alternatively, a therapeutically effective dose is administered solely by intra-arterial injection, but formulated in particles that in themselves deliver the bi-functional molecules over a prolonged period of time. Generally, the local drug delivery devices of this invention contain between about 0.01 mg and about 10.0 mg of a bi-functional molecule. Alternatively, intra-arterial or intravenous injection is generally deliver between about 0.01 mg and about 50 mg of a bi-functional molecule.

### Nanoparticle and Microparticle Formulation

The multivalent molecules disclosed herein may be formulated along with pharmaceutically acceptable carriers and/or polymers, e.g. polyglycolic acid (PGLA), polylactic acid (PLA), PGLA-PLA copolymers, polysaccharides, and/or phospholipids, as spherical particles with diameters of about 100 nm to about 1,000 nm. Such particles are referred to commonly and interchangeably as either microparticles and/or nanoparticles. In such a system, delivery of the therapeutic agent is controlled in several ways: by the elution of the therapeutic agent via diffusion from the particle into the blood; by sequestration and subsequent release of the particles containing therapeutic agents in cells and extracellular matrix upstream of and in the target tissue; and by release of the therapeutic agent via biological breakdown, or degradation, of the particle itself. Details of such processes are described below.

Microparticles containing multivalent molecules are produced by a variety of methods known in the art (Lemke and Hernandez-Trejo, Curr Pharm Biotechnol, 6:3-5, 2005), e.g. the emulsion-solvent evaporation technique (Sengupta et al, Nature, 436:568-572, 2005) or the stable aqueous/aqueous emulsion system (U.S. Pat. No. 6,805,879). Different techniques are chosen based on the chemical, electrical, and hydrophobic properties of a given multivalent molecule. Microparticles are administered by themselves suspended in an appropriate solvent/buffer system (Jiang et al, Adv Drug Deliv Rev, 10:391-410, 2005), by intra-arterial injection (Guzman et al, Circulation, 94:1441-1448, 1996), by PTCA balloon delivery (Kaul et al, Circulation, 107:2551-2554, 2003), or any suitable method. Alternatively, microparticles are incorporated into a device coating such as those described below. Microparticles themselves may incorporate more than one layer, with each layer possessing unique characteristics with regard to formulation and delivery of multivalent molecules.

### Device Coatings

The multivalent molecules used in accordance with the principles of this disclosure are attached to the implantable device by suitable methods known in the art. In some embodiments, the multivalent molecules are attached to the device by way of a polymeric coating having known and controllable release characteristics, being biocompatible when implanted in animals and humans, and being non-thrombogenic when in contact with blood and the vascular system. For example and as discussed herein, the reactants and reaction conditions used to generate the polymer compositions disclosed herein are modified to alter the properties of the final polymer composition. For example, properties such as the diffusion coefficients (e.g., the rate at which the therapeutic agents are able to diffuse through the polymer matrix), the rate of degradation of one or more of the polymer components, and the rate of the release of the multivalent molecules are manipulated by altering the reaction conditions and reagents, and hence the final polymer properties, used to generate the coating polymers.

Two major classes of polymeric coatings are used with implantable devices: biostable, or non-erodable, coatings; and bioabsorbable, or biodegradable, coatings. Examples of biostable coatings are fluorosilicone, silicone co-polymers, polyethylene glycol (PEG), poly(butyl methacrylate), poly(ethylene-co-vinyl acetate), polyvinyl alcohol, polyvinyl acetate, polyvinylpyrrolidone, polyacrylamide, polyacrylic acid, polyhydroxyethyl methacrylate, polyethylene oxide. Examples of bioabsorbable coatings are polyglycolic acid (PGLA), polylactic acid (PLA), PGLA-PLA copolymers, polysaccharides, and phospholipids. In addition, therapeutic agents may be applied directly to implantable devices without polymeric carriers, where the surface of the device is equipped with holes, crevices, micropores, or channels in which the multivalent molecules are sequestered to varying degrees, thus allowing controlled release in vivo.

Delivery of multivalent molecules from biostable coatings occurs via diffusion from the surface and/or interior of the coating into surrounding tissue, interstitial space, or vascular lumen. For bioabsorbable coatings, in vivo hydrolytic degradation of the polymeric coating is an additional mechanism for release of the multivalent molecules, whereby metabolism of the polymeric coating by endogenous enzymes may also play a role (Meyers et al., J. Med. Chem. 2000, 43, 4319-4327). Important factors influencing hydrolytic degradation include water permeability, chemical structure, molecular weight, morphology, glass transition temperature, additives, and other environmental factors such as pH, ionic strength, site of implantation, etc. The duration of sustained delivery can be adjusted from few days up to one year by a person of ordinary skill in the art through proper selection of polymer and fabrication method.

In one embodiment, preparation of coated implantable devices is accomplished by dissolving the dried polymer in a suitable solvent and spin-coating, dipping, or spraying the medical device, typically using, for example, a 5 wt % in 2-propanol solution of the polymer. The selection of other suitable solvents for coating the medical devices will typically depend on the particular polymer as well as the volatility of the solvent.

One method of modulating the properties of the polymer compositions is to control the diffusion coefficient of the one or more polymer coating layers. The diffusion coefficient relates to the rate at which a compound diffuses through a coating matrix. The analyte diffusion coefficient can be determined for the coating compositions of the present invention. Methods for determining diffusion coefficients are described, for example, in U.S. Pat. Nos. 5,786,439 and 5,777,060.

One method for coating a local delivery device includes sequentially applying a plurality of relatively thin outer layers of a coating composition comprising a solvent mixture of polymeric silicone material, a crosslinking agent, and one or more of the multivalent molecules (see, for example, U.S. Pat. No. 6,358,556). The polymeric coatings are cured in situ and the coated, cured prosthesis is sterilized in a step that includes pretreatment with argon gas plasma and exposure to gamma radiation, electron beam, ethylene oxide, and/or steam.

In another embodiment, the polymeric coating is applied as a mixture, solution or suspension of polymeric material and one or more of the therapeutic agents is dispersed in an organic vehicle or a solution or partial solution of such agents in a solvent or vehicle for the polymer and/or the therapeutic agents. Optionally the various multivalent molecules are placed within different polymer layers. The multivalent molecules are dispersed in a carrier material which is variously the polymer, a solvent, or both. In some instances, the coating is applied sequentially in one or more relatively thin layers. In some applications the coating is further characterized as an undercoat and a topcoat. The coating thickness ratio of the topcoat to the undercoat varies with the desired effect and/or the elution system. In an illustrative embodiment of a device having a plurality of coating layers, the coating on the medical device includes one or more base coatings and a top coating (see, for example, U.S. Pat. No. 6,287,285).

In another embodiment, linking agents are used to encapsulate and/or link the multivalent molecules to the polymer matrix or link the various components of the polymer matrix together (e.g., the different polymers that comprise the various coating layers, the bioactive agents in the polymer matrices etc.). Such linking agents include, for example, polyester amide (PEA), polyethylene imine (PEI), avidin-biotin complexes, photolinking, functionalized liposomes, microsponges and microspheres.

In another embodiment, multivalent molecules are modified by chemically linking them to a high molecular weight, water-soluble polymer carrier. This modified multivalent molecules is termed herein an agent-polymer conjugate. One property of the conjugate is that the chemical linkage of the multivalent molecules to the water-soluble polymer can be manipulated to hydrolytically degrade, thereby releasing biologically active multivalent molecules into the environment in which they are placed.

The multivalent molecule-polymer conjugate is incorporated into a controlled release matrix, formulated from a second biocompatible polymer. When implanted into a tissue such as the arterial lumen, the controlled-release matrix releases the multivalent molecule-polymer conjugate which releases free multivalent molecules to treat the area of the tissue in the immediate vicinity of the polymer. The multivalent molecule-polymer conjugates will also diffuse within the tissue, reaching a great distance from the matrix because of their low rate of clearance from the tissue. As the conjugates diffuse, the bond between the polymer and the multivalent molecules slowly degrades in a controlled, prespecified pattern, releasing the active agent into the environment in which they are placed to have its therapeutic effect.

Either synthetic or naturally occurring polymers are useful. While not limited to this group, some types of polymers that are used are polysaccharides (e.g., dextran and ficoll), proteins (e.g., poly-lysine), poly(ethylene glycol), and poly(methacrylates). Different polymers produce different diffusion characteristics.

The rate of hydrolytic degradation, and thus of multivalent molecule release, may be also altered from minutes to months by altering the physico-chemical properties of the bonds between the multivalent molecule and the polymer. While not wishing to be limited to the following types of bonds, multivalent molecules may be bonded to water-soluble polymers using covalent bonds, such as ester, amide, amidoester, and urethane bonds. Ionic conjugates are also used. By changing the nature of the chemical association between water-soluble polymer and multivalent molecules, the half-life of carrier-agent association is varied. This half-life of the conjugate in the environment in which it is placed determines, in part, the rate of release from the polymer and, therefore, the degree of penetration that the conjugate can achieve in the target tissue. Other suitable hydrolytically labile bonds which can be used to link the multivalent molecules to the water soluble polymer include thioester, acid anhydride, carbamide, carbonate, semicarbazone, hydrazone, oxime, iminocarbonate, phosphoester, phosphazene, and anhydride bonds.

The rate of release is also affected by (a) stereochemical control (varying amounts of steric hindrance around the hydrolyzable bonds); (b) electronic control (varying electron donating/accepting groups around the reactive bond, controlling reactivity by induction/resonance); (c) varying the hydrophilicity/hydrophobicity of any optional spacer groups between the therapeutic agent and the polymer; (d) varying the length of the optional spacer groups (increasing length making the bond to be hydrolyzed more accessible to water); and (e) using bonds susceptible to cleavage by soluble blood plasma enzymes.

The properties of the controlled release matrix vary the rate of polymeric conjugate release into the tissue (Dang, et al., Biotechnol. Prog., 8: 527-532, 1992; Powell, et al., Brain Res., 515: 309-311, 1990; Radomsky, et al., Biol. of Repro., 47: 133-140, 1992; Saltzman, et al., Biophys. J., 55: 163-171, 1989; Chemical Engineering Science, 46: 2429-2444, 1991; J. Appl. Polymer Sci., 48: 1493-1500, 1992; Sherwood, et al., BioTechnology, 10: 1446-1449, 1992). Among the variables which affect conjugate release kinetics are: controlled release polymer composition, mass fraction of conjugate within the matrix (increasing mass fraction increases release rate), particle size of multivalent molecule-polymer conjugate within the matrix (increasing particle size increases release rate), composition of polymeric conjugate particles, and polymer size (increasing surface area increases the release rate), and polymer shape of the controlled release matrix. Suitable polymer components for use as controlled-release matrices include poly(ethylene-co-vinyl acetate), poly(DL-lactide), polyglycolide, copolymers of lactide and glycolide, and polyanhydride copolymers.

As discussed in U.S. Pat. No. 6,300,458, hydroxypolycarbonates (HPC) is used as hydroxyl functional polymers that bind therapeutic agents or carbohydrate polymers chemically or via hydrogen bonding. These copolymers have properties attractive to the biomedical area as is or by conversion to the HPC product provided by hydrolysis or by in vivo enzymatic attack. A feature of these polymers is their tendency to undergo surface erosion. To maximize control over the release process, it is useful to have a polymeric system which degrades from the surface and deters the permeation of the agent molecules.

As noted above, the polymer compositions disclosed herein allow for the controlled release of multivalent molecules. This controlled release is modulated by the pH of the environment in which the polymer compositions function. In this context, one embodiment includes the controlled release of the multivalent molecules from a hydrophobic, pH-sensitive polymer matrix (see, for example, U.S. Pat. No. 6,306,422). A polymer of hydrophobic and weakly acidic comonomers is used in the controlled release system. Weakly basic comonomers are used and the active agent is released as the pH drops. For example, a pH-sensitive polymer releases the multivalent molecules when exposed to a higher pH environment as the polymer gel swells. Such release is made slow enough so that the multivalent molecule remains at significant levels for a clinically useful period of time.

Related embodiments provide additional compositions for releasing multivalent molecules using a dual phase polymeric agent-delivery composition. These dual phase polymeric compositions comprise a continuous biocompatible gel phase, a discontinuous particulate phase comprising defined microparticles, and the multivalent molecules to be delivered (see, for example, U.S. Pat. No. 6,287,588). Typically in such embodiments, a microparticle containing a multivalent molecules is entrained within a biocompatible polymeric gel matrix. The multivalent molecule release is contained in the microparticle phase alone or in both the microparticles and the gel matrix. The release of the multivalent molecules is prolonged and the delivery is modulated and/or controlled. In addition, the second agent is loaded in the same or different microparticles and/or in the gel matrix. Alternatively, layered microparticles (or nanoparticles) are produced in which, for example, the inner core consists of a particular polymer carrying the multivalent molecule while an outer layer consisting of the same or a different material either carrying the multivalent molecule and releasing it with different release kinetics (Sengupta et al, Nature, 436:568-572, 2005), or not carrying the multivalent molecule and serves to control its release from the inner core.

Drug-eluting devices of this invention release a plurality of therapeutic agents. These agents are released at a constant rate or at a multi-phasic rate. For example, in one embodiment, the release comprises an initial burst (immediate release) of the therapeutic agents present at or near the surface of the coating layer, a second phase during which the release rate is slower or sometimes no therapeutic agent is released, and a third phase during which most of the remainder of the therapeutic agents is released as erosion proceeds.

All publications and patents cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for treating a cardiac disorder in a patient, said method comprising administering to said patient a molecule, wherein said molecule is capable of binding (i) a coronary tissue marker and (ii) a target cell marker.

2. The method of claim 1, wherein said cardiac disorder is selected from the group consisting of acute myocardial infarction, a chronic ischemic condition, reperfusion injury, chronic heart disease, vulnerable plaques, and cardiac fibrosis.

3. The method of claim 1, wherein said cardiac disorder is acute myocardial infarction.

4. The method of claim 1, wherein said molecule is selected from the group consisting of a bispecific antibody, bispecific F(ab′)₂ fragment, bispecific miniantibody, diabody, triabody, and tetrabody.

5. The method of claim 4, wherein said molecule is a diabody.

6. The method of claim 5, wherein said diabody is a human IgG.

7. The method of claim 1, wherein said coronary tissue marker is selected from the group consisting of matrix, metalloproteinase-1, matrix metalloproteinase-8, fibronectin, osteopontin, TLR4, IL1RL1-b, vascular cell adhesion molecule-1 (VCAM-1), Adam-like decysin-1 (ADAMDEC1), matrix metalloproteinase-9 (MMP-9), CD 163, chemokine (C—X—C motif) ligand-2 (CXCL2), retinoic acid responder-1 (RARRES1), LpPLA2; colony-stimulating factor-2 receptor β (CSF2RB), cathepsin S, chemokine (C—C motif) ligand 18 (CCL 18), spermidine/spermine N1 acetyltransferase, integrin β2 (ITGB2), cystatin B (CSTB), legumain; uncoupling protein 2 (UCP2), cathepsin B, RALGDS, F11 receptor (JAM-A), and ATPase plasma membrane 1 (ATP2B1).

8. The method of claim 1, wherein said target cell marker is present on a target cell is selected from the group consisting of a bone-marrow-derived stem cell, a circulating stem cell, a coronary stem cell, an endothelial progenitor cell, and a hormone/growth factor secreting cell.

9. The method of claim 1, wherein said target cell marker is selected from the group consisting of CD34, CD133, CD14, CXCR4, kinase insert domain receptor (KDR), Flk-1, VE-cadherin, c-Kit, and Sca-1.

10. The method of claim 1, wherein said target cell marker is CD34.

11. The method of claim 1, wherein said molecule is administered by intravenous, intra-arterial, or intramyocardial injection.

12. The method of claim 1, wherein said molecule is administered in the course of percutaneous transluminal coronary angioplasty.

13. The method of claim 1, wherein said molecule is administered using an implantable intra-arterial stent.

14. A molecule capable of binding (i) a coronary tissue marker and (ii) a target cell marker.

15. The molecule of claim 14, wherein said molecule is selected from the group consisting of a bispecific antibody, bispecific F(ab′)₂ fragment, bispecific miniantibody, diabody, triabody, and tetrabody.

16. The molecule of claim 14, wherein said molecule is a diabody.

17. The molecule of claim 16, wherein said diabody is a human IgG.

18. The molecule of claim 14, wherein said coronary tissue marker is selected from the group consisting of matrix metalloproteinases-1, matrix metalloproteinase-8, fibronectin, osteopontin, TLR4, and IL1RL1-b.

19. The molecule of claim 14, wherein said target cell marker is present on a target cell is selected from the group consisting of a bone-marrow-derived stem cell, a circulating stem cell, a coronary stem cell, an endothelial progenitor cell, and a hormone/growth factor secreting cell.

20. The molecule of claim 14, wherein said target cell marker is selected from the group consisting of CD34, CD 133, CD 14, CXCR4, kinase insert domain receptor (KDR), Flk-1, VE-cadherin, c-Kit, and Sea-1.

21. The molecule of claim 14, wherein said target cell marker is CD34.

22. The molecule of claim 14, wherein said molecule comprises a first VH and VL pair and a second VH and VL pair.

23. The molecule of claim 22, wherein said first VH and VL pair has a different binding specificity compared to said second VH and VL pair.

24. The molecule of claim 23, wherein at least one of said first VH and VL pair and said second VH and VL pair is derived from a hybridoma.

25. A pharmaceutical composition comprising the molecule of claim 14 and a pharmaceutically acceptable carrier.

26. The composition of claim 25, wherein said pharmaceutically acceptable carrier is a microparticle.

27. The pharmaceutical composition of claim 25, wherein said composition is suitable for intravenous, intra-arterial, or intramyocardial injection.

28. An implantable device comprising the molecule of claim 14.

29. The device of claim 28, wherein said molecule is coated on said device.

30. The device of claim 28, wherein said molecule is contained within a polymer coating on said device.

31. The device of claim 28, wherein said device is a stent.

32. A cardiovalent molecule for use in treating a cardiac disorder, wherein said cardiovalent molecule is capable of binding (i) a coronary tissue marker and (ii) a target cell marker.

33. The cardiovalent molecule of claim 32, wherein said cardiac disorder is selected from the group consisting of acute myocardial infarction, a chronic ischemic condition, reperfusion injury, chronic heart disease, vulnerable plaques, and cardiac fibrosis.

34. The cardiovalent molecule of claim 33, wherein said cardiac disorder is acute myocardial infarction.

35. The cardiovalent molecule of claim 32, wherein said cardiovalent molecule is selected from the group consisting of a bispecific antibody, bispecific F(ab′)2 fragment, bispecific miniantibody, diabody, triabody, and tetrabody.

36. The cardiovalent molecule of claim 35, wherein said cardiovalent molecule is a diabody.

37. The cardiovalent molecule of claim 35, wherein said diabody is a human IgG.

38. The cardiovalent molecule of claim 32, wherein said coronary tissue marker is selected from the group consisting of matrix metalloproteinase-1, matrix metalloproteinase-8, fibronectin, osteopontin, TLR4, IL1RL1-b, vascular cell adhesion molecule-1 (VCAM-1), Adam-like decysin-1 (ADAMDEC1), matrix metalloproteinase-9 (MMP-9), CD163, chemokine (C—X—C motif) ligand-2 (CXCL2), retinoic acid responder-1 (RARRES1), LpPLA2; colony-stimulating factor-2 receptor β (CSF2RB), cathepsin S, chemokine (C—C motif) ligand 18 (CCL1 8), spermidine/spermine N1 acetyltransferase, integrin β2 (ITGB2), cystatin B (CSTB), legumain; uncoupling protein 2 (UCP2), cathepsin B, RALGDS, F11 receptor (JAM-A), and ATPase plasma membrane 1 (ATP2B1).

39. The cardiovalent molecule of claim 32, wherein said target cell marker is present on a target cell is selected from the group consisting of a bone-marrow-derived stem cell, a circulating stem cell, a coronary stem cell, an endothelial progenitor cell, and a hormone/growth factor secreting cell.

40. The cardiovalent molecule of claim 32, wherein said target cell marker is selected from the group consisting of CD34, CD133, CD14, CXCR4, kinase insert domain receptor (KDR), Flk-1, VE-cadherin, c-Kit, and Sea-1.

41. The cardiovalent molecule of claim 32, wherein said target cell marker is CD34.

42. The cardiovalent molecule of claim 32, wherein said cardiovalent molecule is administered by intravenous, intra-arterial, or intramyocardial injection.

43. The cardiovalent molecule of claim 32, wherein said cardiovalent molecule is administered in the course of percutaneous transluminal coronary angioplasty.

44. The cardiovalent molecule of any of claim 32, wherein said molecule is administered using an implantable intra-arterial stent.
